(19) United States
(12) Patent Application Publication
(10) Pub. No.: US 2011/0184105 A1
(43) Pub. Date: Jul. 28, 2011

(54) Phenylene Oxo-Diester Plasticizers and Methods of Making

(75) Inventors: Jihad Mohammed Dakka, Whitehouse Station, NJ (US); Edmund John Mozeleski, Califon, NJ (US); Lisa Saunders Baugh, Ringoes, NJ (US); Francisco Manuel Benitez, Houston, TX (US); Catherine Anne Faler, Houston, TX (US); Allen David Godwin, Seabrook, TX (US); Jörg Friedrich Wilhelm Weber, Houston, TX (US); Diana S. Smirnova, High Bridge, NJ (US)

(73) Assignee: ExxonMobil Research and Engineering Company, Annandale, NJ (US)

(21) Appl. No.: 12/892,680

(22) Filed: Sep. 28, 2010

Related U.S. Application Data
(60) Provisional application No. 61/277,762, filed on Sep. 29, 2009.

(51) Int. Cl. C08K 5/12; C07C 69/73; C07C 67/08; C09K 3/00

(52) U.S. Cl. 524/287; 560/81; 252/182.23

(57) ABSTRACT

A process for making non-phthalate, 1,2-phenylene oxo-diester plasticizers for polymer compositions, by selectively hydrogenating naphthalene to form a partially hydrogenated naphthalene, oxygenating said partially hydrogenated naphthalene to form phenylene diacids, and esterifying said phenylene diacids with oxo-alcohols to form 1,2-phenylene oxo-diesters. Also a process for making phenylene oxo-diester plasticizers by selectively brominating xylenes to form bisbromomethylbenzene, catalytic carboalkoxylation of the bromo-compound to form phenylene diacetate, followed by transesterification to form the phenylene oxo-diester.

### CROSS-REFERENCE TO RELATED APPLICATIONS

This is a non-provisional application that claims priority to U.S. Provisional Patent Application No. 61/277,762 filed on Sep. 29, 2009, herein incorporated by reference in its entirety.

### FIELD

This disclosure relates to oxo-diesters useful as non-phthalate plasticizers and for a wide range of polymer resins and methods of making such plasticizers.

### BACKGROUND

Plasticizers are incorporated into a resin (usually a plastic or elastomer) to increase the flexibility, workability, or distensibility of the resin. The largest use of plasticizers is in the production of “plasticized” or flexible polyvinyl chloride (PVC) products. Typical uses of plasticized PVC include films, sheets, tubing, coated fabrics, wire and cable insulation and jacketing, toys, flooring materials such as vinyl sheet flooring or vinyl floor tiles, adhesives, sealants, inks, and medical products such as blood bags and tubing, and the like.

Other polymer systems that use small amounts of plasticizers include polyvinyl butyral, acrylic polymers, nylon, polyolefins, polyurethanes, and certain fluoroplastics. Plasticizers can also be used with rubber (although often these materials fall under the definition of extenders for rubber rather than plasticizers). A listing of the major plasticizers and their compatibilities with different polymer systems is provided in “Plasticizers,” A. D. Godwin, in Applied Polymer Science 21st Century, edited by C. D. Craver and C. E. Carraher, Elsevier (2000); pp. 157-175.

Plasticizers can be characterized on the basis of their chemical structure. The most important chemical class of plasticizers is phthalic acid esters, which accounted for about 84% worldwide of PVC plasticizer usage in 2009. However, in the recent past there has been an effort to decrease the use of phthalate esters as plasticizers in PVC, particularly in end uses where the product contacts food, such as bottle cap liners and sealants, medical and food films, or for medical examination gloves, blood bags, and IV delivery systems, flexible tubing, or for toys, and the like. For these and most other uses of plasticized polymer systems, however, a successful substitute for phthalate esters has heretofore not materialized. The majority of PVC plasticizers are various types of mono-, di-, and tri-esters formed by the esterification of acids or anhydrides with C4 to C14 OXO alcohols. Oxo alcohols are primary aliphatic alcohols obtainable through various types of hydroformylation or OXO processes.

One such suggested substitute for phthalates are esters based on cyclohexanoic acid. In the late 1990's and early 2000's, various compositions based on cyclohexanoate, cyclohexanedioates, and cyclohexanepolyoate esters were said to be useful for a range of goods from semi-rigid to highly flexible materials. See, for instance, WO 99/32427, WO 2004/046078, WO 2003/029339, U.S. Application No. 2006-0247461, and U.S. 7,297,738.

Other suggested substitutes include esters based on benzoic acid (see, for instance, U.S. Pat. No. 6,740,254, and also co-pending, commonly-assigned, U.S. Provisional Patent Application No. 61/040,480, filed Mar. 28, 2008 and polyketones, such as described in U.S. Pat. No. 6,777,514; and also co-pending, commonly-assigned, U.S. patent application Ser. No. 12/058,397, filed Mar. 28, 2008. Epoxidized soybean oil, which has much longer alkyl groups (C₁₆ to C₁₈) has been tried as a plasticizer, but is generally used as a PVC stabilizer. Stabilizers are used in much lower concentrations than plasticizers. Co-Pending and commonly-assigned U.S. Provisional Patent Application No. 61/203,626, filed Dec. 24, 2008, discloses triglycerides with a total carbon number of the triester groups between 20 and 25, produced by esterification of glycerol with a combination of acids derived from the hydroformylation and subsequent oxidation of C₃ to C₉ olefins, having excellent compatibility with a wide variety of resins and that can be made with a high throughput.

Typically, the best that has been achieved with substitution of the phthalate ester with an alternative material is a flexible PVC article having either reduced performance or poorer proccessability. Thus, heretofore efforts to make phthalate-free plasticizer systems for PVC have not proven to be entirely satisfactory, and this is still an area of intense research.

SU 487090, which is incorporated by reference herein in its entirety, discloses esterification of 2-carboxymethylbenzoic acid with n-octyl alcohol to form a diester for use as a plasticizer for polyvinyl chloride (PVC). The alcohol used for esterification is not an oxo-alcohol.

GB 1191380, which is incorporated by reference herein in its entirety, discloses preparation of diesters of 1,2-dicarboxylic aromatic acids and oxo-alcohols, but exemplifies only phthalates.

GB 999229, GB 778311 (U.S. Pat. No. 2,832,888) and FR 1179496, which are incorporated by reference herein in their entireties, disclose oxidation of tetralin to 2-carboxymethylbenzoic acid, but fail to suggest esterification of the diacid.

Thus what is needed is a general purpose non-phthalate plasticizer having suitable melting or chemical and thermal stability, glass transition, increased compatibility, good performance and low temperature properties, and a method to make such plasticizer.

### SUMMARY

In one aspect, the present disclosure is directed to oxo-diesters of the formula:

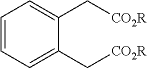

wherein each R is the alkyl residue of one or more C₄ to C₁₄ oxo-alcohols.

In another aspect, the present disclosure is directed to oxo-diesters of the formula:

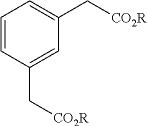

wherein each R is the alkyl residue of one or more C₄ to C₁₄ oxo-alcohols.

In another aspect, the present disclosure is directed to oxo-diesters of the formula:

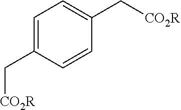

wherein each R is the alkyl residue of one or more C₄ to C₁₄ oxo-alcohols.

In another aspect, the present disclosure is directed to oxo-diesters of the formula:

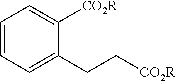

wherein each R is the alkyl residue of one or more C₄ to C₁₄ oxo-alcohols.

In a further aspect of the current disclosure, provided is a process for making phenylene oxo-diesters, comprising: selectively brominating xylene to form bisbromomethylbenzene; carboalkoxlating the bisbromomethylbenzene with a palladium catalyst to form dimethylphenylene diacetate; and transesterifying of the diphenylene diacetate with oxo-alcohols to form phenylene oxo-diesters.

In a further embodiment, the present disclosure is directed to a process for making 1,2-phenylene oxo-diesters, comprising selectively hydrogenating naphthalene to form a partially hydrogenated naphthalene; oxygenating said partially hydrogenated naphthalene to form phenylene diacids; and esterifying said phenylene diacids with oxo-alcohols to form 1,2-phenylene oxo-diesters.

In a further aspect, provided is a mixture of two or more phenylene oxo-diesters chosen from the following formulae and mixtures thereof:

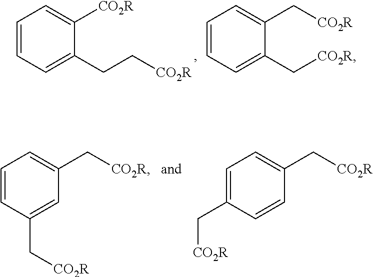

wherein each R is the alkyl residue of one or more C₄ to C₁₄ oxo-alcohols.

In a further embodiment, said selective hydrogenation is conducted by reacting naphthalene with hydrogen at temperatures between 30° C. and 300° C., and a pressure of between 100 kPa to 2000 kPa to form tetralin.

More particularly, said selective hydrogenation is conducted by reacting naphthalene with hydrogen at temperatures between 30° C. and 300° C., and a pressure of between 100 kPa to 2000 kPa to form dihydronaphthalene.

In a further embodiment, said oxidation of tetralin is conducted by reacting tetralin with an oxidant at temperatures between 30° C. and 300° C., to form 1,2-phenylene diacids.

Alternatively, said oxidation of dihydronaphthalene is conducted by reacting dihydronaphthalene with an oxidant at temperatures between 30° C. and 300° C., to form 1,2-phenylene diacids.

In a further embodiment, said esterification of 1,2-phenylene diacids is conducted by reacting said 1,2-phenylene diacids with C₄ to C₁₄ oxo-alcohols at temperatures between 100° C. and 250° C., to form 1,2-phenylene oxo-diesters.

In another embodiment, the present invention is directed to a polymer composition comprising a polymer and at least one phenylene oxo-diester selected from the following formulae and mixtures thereof:

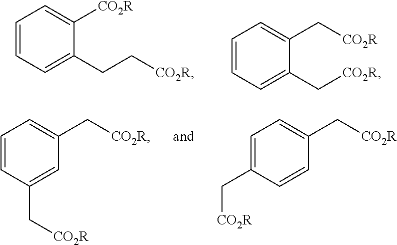

wherein each R is the alkyl residue of one or more C₄ to C₁₄ oxo-alcohols.

Advantageously, the polymer is selected from the group consisting of vinyl chloride resins, polyesters, polyurethanes, ethylene-vinyl acetate copolymer, rubbers, poly(meth)acrylics and combinations thereof, such as a polymer blend of polyvinyl chloride with an ethylene-vinyl acetate copolymer; or a polymer blend of polyvinyl chloride with a polyurethane; or a polymer blend of polyvinyl chloride with an ethylene-based polymer, and more advantageously, the polymer is polyvinyl chloride.

### DETAILED DESCRIPTION

All numerical values within the detailed description and the claims herein are modified by “about” or “approximately” the indicated value, and take into account experimental error and variations that would be expected by a person having ordinary skill in the art.

There is an increased interest in developing new plasticizers that are non-phthalates and which possess good plasticizer performance characteristics but are still competitive economically. The present disclosure is directed towards non-phthalate plasticizers that can be made from low cost feeds and that may employ fewer manufacturing steps in order to potentially lower manufacturing costs associated with plasticizer production. One route into non-phthalate plasticizers is to produce diacids from naphthalene feeds using hydrogenation followed by oxidation. The di-acids of the hydrogenation and the oxidation step(s) may be esterified with C4 to C14 alcohols to form oxo-esters. The C4 to C14 alcohols may be primary aliphatic alcohols and may be branched or linear. One advantageous source of C4 to C14 alcohols of the present disclosure is through the OXO process. Another route to forming non-phthalate oxo-ester plasticizers is chain bromination of xylene followed by conversion to esters by carboalkoxylation, then to oxo-esters by transesterification.

An “oxo-diester” is a compound having two functional ester moieties within its structure that are derived from esterification of a di-acid compound with an oxo-alcohol.

An “oxo-alcohol” is an organic alcohol, or mixture of organic alcohols, which is prepared by hydroformylating an olefin, followed by hydrogenation to form the alcohols. Typically, the olefin is formed by light olefin oligomerization over heterogeneous acid catalysts, which olefins are readily available from refinery processing operations. The reaction results in mixtures of longer-chain, branched olefins, which subsequently form longer chain, branched alcohols, as described in U.S. Pat. No. 6,274,756, incorporated herein by reference in its entirety. Another source of olefins used in the OXO process are through the oligomerization of ethylene, producing linear alpha olefins. producing mixtures of predominately straight chain alcohols with lesser amounts of lightly branched alcohols.

“Hydroformylating” or “hydroformylation” is the process of reacting a compound having at least one carbon-carbon double bond (an olefin) in an atmosphere of carbon monoxide and hydrogen over a cobalt or rhodium catalyst, which results in addition of at least one aldehyde moiety to the underlying compound. U.S. Pat. No. 6,482,972, which is incorporated herein by reference in its entirety, describes the hydroformylation (oxo) process.

“Hydrogenating” or “hydrogenation” is addition of hydrogen (H₂) to a double-bonded functional site of a molecule. Conditions for hydrogenation of an aldehyde are well-known in the art and include, but are not limited to temperatures of 0-300° C., pressures of 1-500 atmospheres, and the presence of homogeneous or heterogeneous hydrogenation catalysts such as Pt/C, Pt/Al₂O₃ or Pd/Al₂O₃.

“Oxidizing” or “oxidation” is addition of at least one oxygen atom to organic compound, such as in the present case, addition of an oxygen atom to the aldehyde moieties of a di-aldehyde to form the corresponding di-carboxylic acid. Oxygen for the reaction can be provided by air or oxygen-enriched air. Conditions for oxidation of an aldehyde are well known in the art, and include, but are not limited to temperatures of 0-300° C., pressures of 1-500 atmospheres, and the presence or absence of homogeneous or heterogeneous oxidation catalysts such as transition metals.

“Esterifying” or “esterification” is reaction of a carboxylic acid moiety with an organic alcohol moiety to form an ester linkage. Esterification conditions are well-known in the art and include, but are not limited to, temperatures of 0-300° C., and the presence or absence of homogeneous or heterogeneous esterification catalysts such as Lewis or Brønsted acid catalysts.

“Tranesterifying” or “transesterification” is reaction of an ester with an organic alcohol moiety to form a different ester. Transesterification conditions are known in the art and include, but are not limited to, temperatures of 100-200° C. and the presence of acid or base catalysts.

This disclosure is related to a potential route to non-phthalate plasticizers using naphthalene as a feedstock, which is selectively hydrogenated to form tetrahydronaphthalene (tetralin) or dihydronaphthalene, and then partially oxidized to form di-acids, as illustrated below.

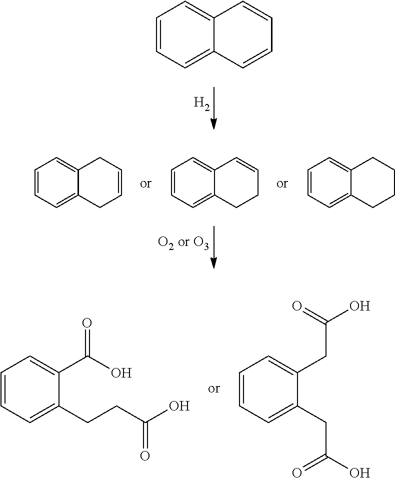

One aspect of the present disclosure is a process for making 1,2-phenylene oxo-diesters, comprising selectively hydrogenating naphthalene to form a partially hydrogenated naphthalene, oxygenating said partially hydrogenated naphthalene to form phenylene diacids, and esterifying said phenylene diacids with oxo-alcohols to form 1,2-phenylene oxo-diesters.

Selective hydrogenation of naphthalene to tetralin is known in the art and is commercially practiced. EP 0087597, which is incorporated by reference herein in its entirety, discloses catalytic hydrogenation of naphthalene to give tetralin at an elevated temperature with hydrogen on a nickel supported catalyst carried out in a solvent at 150 to 250° C. and under a pressure of 1 to 20 bar. U.S. Pat. No. 4,313,017, which is incorporated by reference herein in its entirety, discloses reacting a polynuclear aromatic, such as naphthalene, over a zinc titanate catalyst under conditions to selectively hydrogenate the reactant. The zinc titanate catalyst employed in the present invention is modified with a promoter to improve the selective hydrogenation process.

Alternatively, naphthalene can be selectively hydrogenated to form 1,2-dihydronaphthalene and/or 1,4-dihydronaphthalene. U.S. Pat. No. 5,424,264, which is incorporated by reference herein in its entirety, discloses catalysts and a process for partially hydrogenating polycyclic and monocyclic aromatic hydrocarbons such as benzene, naphthalenes, biphenyls, and alkylbenzenes to produce the corresponding cycloolefins. The catalyst is a hydrogenation catalyst comprising ruthenium on a composite support, and cycloolefins are produced in high yield and with high selectivity.

Selective hydrogenation can be conducted by reacting naphthalene with hydrogen at temperatures between 30° C. and 300° C., and under a hydrogen pressure of between 100 kPa to 2000 kPa (1 to 20 bar) so as to form tetralin, 1,2-dihydronaphthalene and/or 1,4-dihydronaphthalene.

Subsequently, any of tetralin, 1,2-dihydronaphthalene and/or 1,4-dihydronaphthalene is oxidized to form 1,2-diacids of one or both of the following formulae:

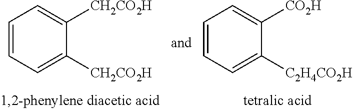

by reacting with an oxidant, such as oxygen, ozone or air, in the presence of a catalyst at temperatures from 30° C. to 300° C., even from 60° C. to 200° C. Catalysts such as vanadium pentoxide, as disclosed in GB 999,229, or in the presence of a chromium exchanged cation-exchange resin catalyst, as disclosed in U.S. Pat. No. 4,473,711, both of which are incorporated by reference herein in their entireties, can be used.

In an alternative process embodiment, the present disclosure is related to a route to non-phthalate plasticizers using xylene as a feedstock. A single xylene isomer, or a mixture of isomers, can be selectively brominated to bisbromomethylbenzene, then carboalkoxylated to a diester as illustrated below.

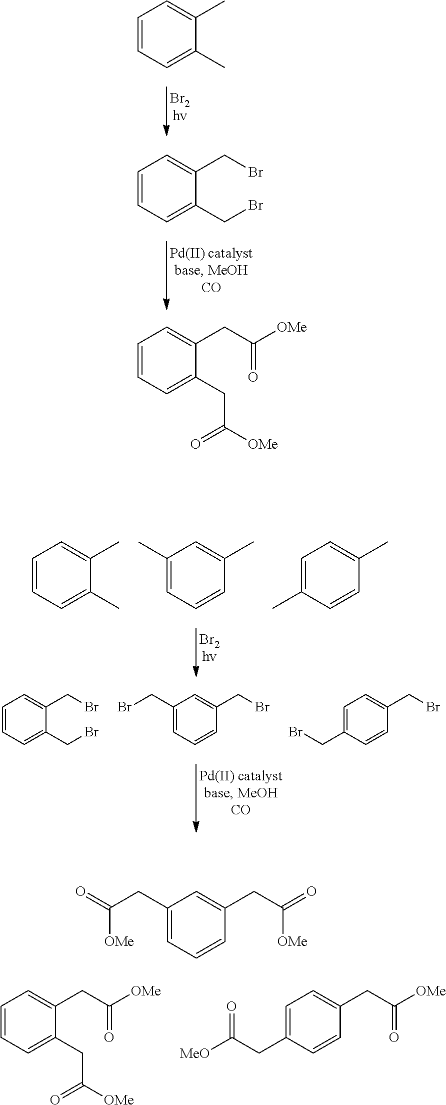

In one form of this alternative process embodiment, a process for making 1,2-phenylene oxo-diesters, includes selectively brominating o-xylene to form 1,2-bisbromomethylbenzene; carboalkoxylating said 1,2-bisbromomethylbenzene with a palladium catalyst to form dimethylphenylene diacetate; and transesterifying the diphenylene diacetate with oxo-alcohols to form 1,2-phenylene oxo-diesters.

In another form of this alternative process embodiment, a process for making 1,3-phenylene oxo-diesters, includes selectively brominating m-xylene to form 1,3-bisbromomethylbenzene; carboalkoxylating said 1,3-bisbromomethylbenzene with a palladium catalyst to form dimethylphenylene diacetate; and transesterifying said diphenylene diacetate with oxo-alcohols to form 1,3-phenylene oxo-diesters.

In yet another form of this alternative process embodiment, a process for making 1,4-phenylene oxo-diesters, includes selectively brominating p-xylene to form 1,4-bisbromomethylbenzene; carboalkoxylating said 1,4-bisbromomethylbenzene with a palladium catalyst to form dimethylphenylene diacetate; and transesterifying said diphenylene diacetate with oxo-alcohols to form 1,4-phenylene oxo-diesters.

In yet another form of this alternative process embodiment, a process for making phenylene oxo-diesters, includes selectively brominating xylene to form bisbromomethylbenzene; carboalkoxlating said bisbromomethylbenzene with a palladium catalyst to form dimethylphenylene diacetate; and transesterifying said diphenylene diacetate with oxo-alcohols to form phenylene oxo-diesters, wherein the xylene is a mixture of two or more of o-xylene, m-xylene, or p-xylene.

The formation of the desired oxo-alcohols to be used for esterification can be accomplished by producing branched aldehydes by hydroformylation of C₃ to C₁₃ olefins that in turn have been produced by propylene and/or butene oligomerization over solid phosphoric acid or zeolite catalysts. The resulting C₄ to C₁₄ aldehydes can then be recovered from the crude hydroformylation product stream by fractionation to remove unreacted olefins. These C₄ to C₁₄ aldehydes can then be hydrogenated to alcohols (oxo-alcohols), which can then be used to esterify the tetralin to form plasticizers. Single carbon number alcohols can be used in the esterification, or differing carbon numbers can be use to optimize product cost and performance requirements.

Alternatively, the oxo-alcohols can be prepared by aldol condensation of shorter-chain aldehydes to form longer chain aldehydes, as described in U.S. Pat. No. 6,274,756, followed by hydrogenation to form the oxo-alcohols. In some embodiments of the disclosure, the oxo-alcohols used to esterify the diacids have an average branching of from 0.2 to 4.0 branches per molecule, more advantageously from 0.8 to 3.0 branches per molecule. In one embodiment, the average branching may range from 1.0 to 2.4 branches per molecule. In another embodiment, C₅ to C₈ alcohols are used having an average branching of from 1.2 to 2.2 branches per molecule, advantageously from 1.2 to 2.0, more advantageously from 1.2 to 1.8 branches per molecule. In other embodiments, the average branching per molecule of the oxo-alcohols used to esterify the diacids will be from 1.2 to 1.6. In yet other embodiments, the oxo-alcohols used may have the branching properties of their precursor olefins described in International Patent Applications WO03/082778 and WO03/082781, United States Patent Application US2005/0014630, or U.S. Pat. No. 7,507,868, all herein incorporated by reference. Tables 1 and 1a below provides typical characteristics of oxo-alcohols.

TABLE 1
Examples of OXO-Alcohols for Use in Preparations of OXO-Diesters.

| | | Average | Average | Examples of |
|---|---|---|---|---|
| | Brand or | Carbon | branches per | Commercial |
| Chemical Name | Other name | Number¹ | Molecule² | Sources |
| Isoheptanol | Exxal ™ 7 | 7.1 | 1.2 | ExxonMobil Chemical |
| Isooctanol | Exxal ™ 8 | 8.1 | 1.6 | ExxonMobil Chemical |
| Isononanol | Exxal ™ 9 | 9.2 | 2.1 | ExxonMobil Chemical |
| Isodecanol | Exxal ™ 10 | 10.0 | 2.1 | ExxonMobil Chemical |
| Isotridecanol | Exxal ™ 13 | 12.7 | 2.9 | ExxonMobil Chemical |
| 2-ethylhexanol | 2-EH | 8.0 | 1.0 | BASF, Eastman |
| 2-propylheptanol | 2PH, | 10.0 | 1.1 | BASF, Evonik |
| | propylheptanol | | | |
| Isononanol | | 9.0 | 1.2-1.5 | BASF, Evonik |
| Isotridecanol | Tridecanol | 13.0 | 2.3-2.5 | BASF, Evonik, Sasol |

¹Average Carbon Number determined by Gas Chromatography and by ¹H NMR
²Average branches per molecule determined by ¹H NMR measurements

TABLE 1a
Other Properties of Typical OXO-Alcohols.

| | Exxal 7 | Exxal 8 | Exxal 9 | Exxal 10 | Exxal 13 |
|---|---|---|---|---|---|
| Chemical Name | isoheptanol | isooctanol | isononanol | isodecanol | isotridecanol |
| Approx/Avg MW | 116 | 130 | 144 | 158 | 200 |
| Hydroxyl No, mg | 476 | 429 | 380 | 353 | 285 |
| KOH/g | | | | | |
| Distillation Range, ° C. | 169-182 | 186-193 | 204-216 | 231-238 | 253-265 |
| Color, Pt/Co | 5 | 5 | 5 | 5 | 5 |
| Carbonyl Number, mg | 0.1 | 0.1 | 0.1 | 0.1 | 0.1 |
| KOH/g | | | | | |
| Water Content, wt. % | 0.05 | 0.05 | 0.05 | 0.05 | 0.05 |
| Viscosity at 20 C, cSt | 10 | 13 | 17 | 21 | 17 |
| Flash Point, ° C. | 70 | 76 | 90 | 102 | 126 |
| Vapor Pressure at 100 C., | 6.28 | 3.49 | 1.56 | 1.08 | 0.20 |
| kPa | | | | | |
| calculated | | | | | |

The resulting C₄ to C₁₄ alcohols can be used individually or together in alcohol mixtures having different chain lengths, to make mixed carbon number esters to be used as plasticizers. This mixing of carbon numbers and levels of branching can be advantageous to achieve the desired compatibility with PVC.

One non-limiting exemplary oxo-alcohol of the present disclosure is 2-propyl heptanol produced by reacting butene in the OXO process to give a C5 aldehyde. The C5 aldehyde is then dimerized to a C10 unsaturated aldehyde, which is then hydrogenated to 2-propyl heptanol. Another non-limiting exemplary oxo-alcohol of the present disclosure is 2-ethyl hexanol produced by reacting propylene through the OXO process to butanal followed by the dimerization of the butanal to the C8 unsaturated aldehyde followed by hydrogenation of the C8 unsaturated aldehyde to 2-ethyl hexanol.

Generally, the oxo-diester plasticizers of the present disclosure will be of the formula

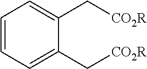

or of the formula

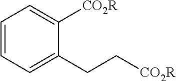

or of the formula

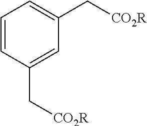

or of the formula:

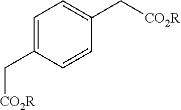

wherein each R is the alkyl residue of one or more C₄ to C₁₄ oxo-alcohols, or mixtures of these oxo-diesters.

The oxo-diester plasticizers of the present application find use in a number of different polymers, such as vinyl chloride resins, polyesters, polyurethanes, ethylene-vinyl acetate copolymers, rubbers, poly(meth)acrylics and mixtures thereof.

For example, the polymer can be a blend of polyvinyl chloride with an ethylene-vinyl acetate copolymer, or a blend of polyvinyl chloride with a polyurethane, or a blend of polyvinyl chloride with an ethylene-based polymer. Advantageously, the polymer is polyvinyl chloride.

### EXAMPLES

### Example #1

### Synthesis of 1,2-bisbromomethylbenzene

A vigorously stirred suspension of o-xylene (1 eq.) and water was exposed to light from an incandescent 300 W light bulb. Bromine (3 eq.) was added drop wise. The resulting red/orange mixture was stirred until disappearance of the bromine color. Ethyl acetate was added to the reaction and the layers separated. The organic solution was dried over MgSO₄ and concentrated to a clear oil which solidifies upon standing. The product was obtained as a mixture of 82% 1,2-bisbromomethyl benzene, 17% 1-(bromomethyl)-2-methylbenzene, and trace amounts of ring-brominated xylene. Melting point and 250 MHz NMR spectra were consistent with an authentic sample.

### Example #2

### Synthesis of 2,2′-(1,2-phenylene)diacetonitrile

To a solution of sodium cyanide (2.0 g, 40.8 mmol) in 25 mL water, a suspension of α,α′-dibromo-o-xylene (4.3 g, 16.3 mmol) in 50 mL ethanol was added. The mixture was refluxed for 2 h and the clear solution cooled to ambient temperature, then concentrated. The aqueous solution was extracted with three portions of methylene chloride and the combined organic layers dried over MgSO₄, filtered and concentrated to give the dinitrile in 70% crude yield: R_{f} 0.23 (30:70 acetone/hexane); IR (cm⁻¹): 3362, 3067, 2928, 2250, 1625, 1495, 1455, 1417, 751; ¹H NMR (250 MHz, C₆D₆) δ 2.52 (s, 4H), 6.81 (m, 2H), 6.94 (m, 2H).

### Example #3

### Synthesis of 2,2′-(1,2-phenylene)diacetic acid

The above dinitrile was dissolved in 50 mL concentrated HCl and refluxed for 3 h. Water (30 mL) was added and the reaction heated overnight, then cooled and washed with ether. The organic layer was extracted twice with sodium carbonate. Combined aqueous layers were acidified and extracted with ether, which was dried (MgSO₄), filtered and concentrated. The diacid was obtained as a pale yellow solid in 56% yield: mp 123-125° C.; ¹H NMR (250 MHz, DMSO-D₆) δ 3.58 (s, 4H), 7.20 (s, 4H), 12.34 (br s, 2H); ¹³C NMR (63 MHz, DMSO-D₆) 37.1 (2C), 126.8 (2C), 130.6 (2C), 134.1 (2C), 172.4 (2C).

### Example #4

### Synthesis of 2,2′-(1,3-phenylene)diacetonitrile

To a solution of sodium cyanide (460 mg, 9.4 mmol) in 6 mL water, a suspension of α,α′-dibromo-m-xylene (1.0 g, 3.7 mmol) in 12 mL ethanol was added. The mixture was refluxed for 1.5 h and the clear solution cooled to ambient temperature, then concentrated. The aqueous solution was extracted with two portions of methylene chloride and the organic layer dried over MgSO₄, filtered and concentrated. ¹H NMR (250 MHz, C₆D₆) δ 2.68 (s, 4H), 6.55 (s, 1H), 6.70 (m, 2H), 6.80 (m, 1H); ¹³C NMR (63 MHz, C₆D₆) 22.5 (2C), 117.5 (2C), 127.2 (2C), 127.5, 129.5, 131.5 (2C).

### Example #5

### Synthesis of 2,2′-(1,3-phenylene)diacetic acid

Obtained from the above 1,3-phenylenediacetonitrile following the procedure described for 2,2′-(1,2-phenylene)diacetic acid: mp 125-134° C.; ¹H NMR (250 MHz, CD₃OD) δ 3.57 (s, 4H), 7.22 (in, 4H); ¹³C NMR (63 MHz, CD₃OD) 41.7 (2C), 128.8 (2C), 129.4, 131.2, 135.9 (2C), 175.3 (2C).

### General Procedure for Esterification

Into a four-necked, 1000 mL round bottom flask equipped with an air stirrer, nitrogen inductor, thermometer, Dean-Stark trap and chilled water cooled condenser were added x moles of diacid (typically either 1,2-phenylene diacetic acid or tetralic acid) and y moles of oxo-alcohol (as specified in the specific Examples). The alcohols used may be a mixture of alcohols having n and m carbons (n and m may the same or different and are branched or mixtures of linear and branched alcohols). The Dean-Stark trap was filled with the lighter boiling alcohols to maintain the same molar ratio of alcohols in the reaction flask. The reaction mixture was heated to 220° C. with air stirring under a nitrogen sweep. The water collected in the Dean-Stark trap was drained frequently and monitored over the course of the reaction to determine conversion. The reaction mixture was heated for the amount of time sufficient to achieve nearly complete conversion to the di-ester. The excess alcohols plus some monoesters were removed by distillation. After distillation, higher product purity was observed in all Examples. Gas chromatography analysis on the products was conducted using a Hewlett-Packard 5890 GC equipped with a HP6890 autosampler, a HP flame-ionization detector, and a J&W Scientific DB-1 30 meter column (0.32 micrometer inner diameter, 1 micron film thickness, 100% dimethylpolysiloxane coating). The initial oven temperature was 60° C.; injector temperature 290° C.; detector temperature 300° C.; the temperature ramp rate from 60 to 300° C. was 10° C./minute with a hold at 300° C. for 14 minutes. The calculated %'s reported for products were obtained from peak area, with an FID (flame ionization) detector uncorrected for response factors.

### Example #6

### Synthesis of Oxo-C₉ diester of 1,2-phenylene diacetic acid

The general esterification procedure described above was followed using 25.5 g (0.1313 mol) of 1,2-phenylene diacetic acid and 113.8 g (0.7879 g) of ExxonMobil Chemical Co. Exxal C₉ alcohol (isomeric mixture). The mixture was heated at 196-215° C. for a total of 5 hours. The selectivity observed in the crude product was 2.6% monoester and 97.3% diester by GC. Following removal of residual monoester and alcohols by distillation, the crude product was treated with decolorizing charcoal (1 wt %) by stirring at room temperature for 2 hours, then filtered. The diester was isolated as the distillation residue in 99.2% purity.

### Example #7

### Synthesis of dimethyl 2,2′-(1,2-phenylene)diacetate

Bisbromomethylbenzene (1 eq.), Pd(PPh₃)₂Cl₂ (0.1 eq.), and potassium carbonate (3 eq.) were combined in a 4:1 mixture of THF:MeOH under an N₂ atmosphere. The solution was purged with CO, and then allowed to stir under a balloon of CO for 18 h. Water was added and the mixture concentrated under reduced pressure. The solution was extracted with ethyl acetate, and then the organic layer was washed with brine and dried over MgSO₄. Concentration under reduced pressure gave an oily residue, which was purified by column chromatography (30:70 acetone:hexane) to give the diacetate in 80% yield. R_{f} 0.64 (30:70 acetone/hexane); ¹H NMR (250 MHz, C₆D₆) δ 3.24 (s, 6H), 3.54 (s, 4H), 6.99 (m, 2H), 7.06 (m, 2H); ¹³C NMR (63 MHz, C₆D₆) 38.9 (2C), 51.4 (2C), 127.6 (2C), 131.1 (2C), 133.8 (2C), 171.1.

### Example #8

### Synthesis of dinonyl 2,2′-(1,2-phenylene)diacetate (Oxo-C₉ diester of 1,2-phenylene diacetic acid)

The above dimethylphenylene diacetate (1 eq.) was dissolved in Oxo-C₉ alcohol (2.2 eq) and a catalytic amount of sulfuric acid was added. Methanol was distilled from the mixture. IR (cm⁻¹): 2957, 1737, 1463, 1250, 1157, 994, 737; ¹H NMR (250 MHz, C₆D₆) δ 0.72-1.13 (m, 34H), 3.69 (s, 4H), 3.96 (m, 4H), 7.00 (m, 2H), 7.13 (m, 2H).

### Example #9

### Synthesis of 1,4-dihydronaphthalene

Stabilized sodium Na-SG(1) (4 eq.) was suspended in THF and cooled to 0° C. t-Amyl alcohol (4 eq.) was added slowly, followed by addition of naphthalene (1 eq.). The reaction was carefully quenched after 2 h with MeOH/H₂O. The mixture was then filtered and concentrated under reduced pressure to give the reduced naphthalene in 51% yield. ¹H NMR (250 MHz, C₆D₆) δ 3.15 (s, 4H), 5.75 (s, 2H), 6.92 (m, 2 H), 7.04 (m, 2H).

### Example #10

### Synthesis of Oxo-C₇ diester of 1,2-phenylene diacetic acid

The general esterification procedure described above was followed using 25.5 g (0.1313 mol) of 1,2-phenylene diacetic acid and 91.7 g (0.7879 g) of ExxonMobil Chemical Co. Exxal C₇ alcohol (isomeric mixture) (CAS Registry Number—70914-20-4). Exxal C₇ alcohol is a mixture of C6-C8 alcohols, and predominately C7 branched aliphatic alcohols. The mixture was heated at 153-167° C. for a total of 6 hours. The selectivity observed in the crude product was 3.5% monoester and 96.4% diester by GC. Following removal of residual monoester and alcohols by distillation, the crude residual product was treated with decolorizing charcoal (1 wt %) by stirring at room temperature for 2 hours, then filtered. The diester was isolated as the distillation residue in 99.8% purity.

### Example #11

### Synthesis of Oxo-C₉ diester of tetralic acid

The general esterification procedure described above was followed using 10.0 g (0.0515 mol) of tetralic acid and 89.2 g (0.6179 g) of ExxonMobil Chemical Co. Exxal C₉ alcohol (isomeric mixture). The mixture was heated at 199-207° C. for a total of 12 hours. The selectivity observed in the crude product was 3.4% monoester and 96.6% diester by GC. Following removal of residual monoester and alcohols by distillation, the diester was isolated as the distillation residue in 99.1% purity.

### Example #12

### Synthesis of Oxo-C₉ diester of homophthalic acid

In a 2 L 3-neck round bottom flask equipped with reflux condenser and Dean Stark trap, was added 958.1 g of Exxal 9 alcohol and 416 g of homophthalic acid (also known as alpha-carboxy-o-toluic acid or 2-carboxyphenyl acetic acid or 2-carboxylbethyl benzoic acid). Under a nitrogen atmosphere the reaction temperature was slowly increased. When the reaction temperature reached 170° C., 1.71 grams tetraisopropyl titanate esterification catalyst diluted with 20 mL of Exxal 9 alcohol was added dropwise. The temperature was slowly increased to 220° C., with the water of reaction collected in a Dean Stark track. After about 5 hrs of reaction time, when the quantity of the collected water was approaching theoretical calculations, a 0.7 g sample was removed and tested for acid conversion by titration. The conversion at this point was calculated to be 99.94% based on conversion of homophthalic acid. The reaction was cooled to 90° C., and 10 grams of Na₂CO₃, 0.25 g of Darco S51-FF and 0.15 grams dicalite filtration aid were added. After stirring for 30 minutes, the vacuum was slowly decreased to 80 mbar for another 30 minutes. The reaction pressure was slowly increased to atmospheric pressure, the temperature cooled to room temperature, and the reaction mixture filtered over a small bed of dicalite filter aid. The excess alcohol was removed in a separate step by steam stripping, under partial vacuum, at 165° C. Gas Chromatography of the reaction product yielded a moderately broad peak with a retention time of 20.5 minutes, consistent with expectations. Infrared analysis of the reaction product yielded the following results: ¹H NMR in CDCl₃: CH3 resonances centered about 0.856 ppm; CH₂, CH resonances between 1.0 and 1.8 ppm; CH₂ (carboxymethylene) at 4.0 ppm; OCH₂ multiple peaks centered at 4.06 and at 4.25 ppm; aromatic resonances at 7.23 (doublet), 7.34 (triplet), 7.46 (triplet), and 8.00 (doublet) ppm.

### Example #13

### Synthesis of Oxo-C₁₀ diester of tetralic acid

The general esterification procedure described above was followed using 10.0 g (0.0515 mol) of tetralic acid and 71.9 g (0.454 g) of ExxonMobil Chemical Co. Exxal C₁₀ alcohol (isomeric mixture). The mixture was heated at 192-220° C. for a total of 6 hours. The selectivity observed in the crude product was 6.1% monoester and 93.7% diester by GC. Following removal of residual monoester and alcohols by distillation, the diester was isolated as the distillation residue in 99.1% purity.

### Example #14

### Synthesis of Oxo-C₉ Diester of 1,3-phenylenediacetic acid

Into a four necked 1 liter round bottom flask equipped with a chilled water condenser, Dean-Stark trap, thermometer and nitrogen inductor were added 1,3-phenylene diacetic acid (127.0 g, 0.654 mol, Aldrich Chemical Co.) and ExxonMobil Chemical Co. Exxal C₉ alcohol (isomeric mixture, 377.8 g, 2.616 mol). After 1 hour of heating at 220° C., toluene (20.0 g, 0.217 mol) was added to maintain a reaction mixture temperature below 220° C. The reaction mixture was heated with air stirring at 195-219° C. for 3 hours. The theoretical weight of water byproduct was obtained after 2 hours heating. The product was distilled overhead under high vacuum (215-219° C./0.10 mm, 99.98% purity).

### Example #15

### Synthesis of Oxo-C₉ Diester of 1,4-phenylenediacetic acid

The same procedure as the proceeding Example was followed using 1,4-phenylene diacetic acid (102.2 g, 0.5263 mol, Aldrich Chemical Co.), Exxal 9 alcohols (304.0 g, 2.106 mol) from ExxonMobil Chemical Company, and toluene (9.8 g, 0.106 mol). The reaction mixture was heated for a total of 7 hours at 190-210° C.; the theoretical amount of water was obtained after 1.5 hours of heating. The product was distilled (220-224° C./0.10 mm). The heart cuts were combined with sample purity of 99.6%.

### Example #16

### Preparation of Feedstock-Representative Ortho:Meta:Para Oxo-C₉ Diester Phenylenediacetic Acid Blend

To provide a material representative of a typical mixed xylenes stream, a 25:53:22 by weight blend of the diesters prepared in Examples 6, 14, and 15 was prepared. This blend was evaluated alongside its pure components as described in subsequent Examples.

### Example #17

### Viscosity, Volatility, and Glass Transition Property Study of Neat Diesters

Thermogravimetric Analysis (TGA) was conducted on the neat diesters using a TA Instruments AutoTGA 2950HR instrument (25-600° C., 10° C./min, under 60 cc N₂/min flow through furnace and 40 cc N₂/min flow through balance; sample size 10-20 mg). Differential Scanning calorimetry (DSC) was also performed, using a TA Instruments 2920 calorimeter fitted with a liquid N₂ cooling accessory. Samples were loaded at room temperature and cooled to −130° C. at 10° C./min and analyzed on heating to 75° C. at a rate of 10° C./min. Table 2 below provides volatility, viscosity, and glass transition (T_{g}) properties of the neat esters. T_{g}s given in Table 2 are midpoints of the second heats (unless only one heat cycle was performed, in which case the first heat T_{g}, which were typically in very close agreement, is given). Kinematic Viscosity (KV) was measured at 20° C. according to ASTM D-445-20, the disclosure of which is incorporated herein by reference. Cone-and-plate viscosity was measured in centipoise (cP) using an Anton Paar (25 mm) viscometer; sample size ˜0.1 mL. Comparative data for a common commercial plasticizer (diisononyl phthalate; Jayflex® DINP, ExxonMobil Chemical Co.) is also included.

TABLE 2

| | TGA 1% | TGA 5% | TGA 10% | TGA Wt | | KV | Viscosity |
|---|---|---|---|---|---|---|---|
| | Wt Loss | Wt Loss | Wt Loss | Loss at | DSC T_{g} | (20° C., | (20° C., |
| Ex. No. | (° C.) | (° C.) | (° C.) | 220° C. (%) | (° C.) | mm²/sec) | cP) |
| DINP | 184.6 | 215.2 | 228.5 | 6.4 | −79.1 | 96.81 | 99.2 |
| 6^{a} | 189.9 | 222.1 | 237.8 | 4.5 | −84.0 | 52.39 | — |
| | (185.8) | (220.1) | (235.8) | (5.0) | (−79.2) | | |
| | (190.3) | (226.6) | (243.4) | (3.7) | (−79.8) | | |
| 10 | 161.1 | 195.9 | 211.3 | 14.7 | −90.2 | 28.32 | — |
| 11 | 185.8 | 220.1 | 235.8 | 5.0 | −79.2 | — | 76.77 |
| 13 | 202.1 | 236.1 | 252.0 | 2.3 | −77.3 | — | — |
| 14 | 197.9 | 230.3 | 246.3 | 3.1 | −86.8 | — | 44.16 |
| 15 | 199.1 | 233.4 | 249.9 | 2.7 | −86.1 | — | 46.98 |
| 16 | 192.9 | 225.7 | 243.1 | 3.9 | −86.1 | — | — |

— = Data not taken.
^{a}Data in parentheses is for two repeat syntheses, each 99.1% purity.

### Example #18

### Procedure for the Use of Esters to Plasticize poly(vinyl Chloride)

A 5.85 g portion of the ester sample (or comparative commercial plasticizer DINP) was weighed into an Erlenmeyer flask which had previously been rinsed with uninhibited tetrahydrofuran (THF) to remove dust. A 0.82 g portion of a 70:30 by weight solid mixture of powdered Drapex® 6.8 (Crompton Corp.) and Mark® 4716 (Chemtura USA Corp.) stabilizers was added along with a stirbar. The solids were dissolved in 117 mL uninhibited THF. Oxy Vinyls® 240F polyvinyl chloride (PVC) (11.7 g) was added in powdered form and the contents of the flask were stirred overnight at room temperature until dissolution of the PVC was complete. The clear solution was poured evenly into a flat aluminum paint can lid (previously rinsed with inhibitor-free THF to remove dust) of dimensions 7.5″ diameter and 0.5″ depth. The lid was placed into an oven at 60° C. for 2 hours with a moderate nitrogen purge. The pan was removed from the oven and allowed to cool for a ˜5 min period. The resultant clear film was carefully peeled off of the aluminum, flipped over, and placed back evenly into the pan. The pan was then placed in a vacuum oven at 70° C. overnight to remove residual THF. The dry, flexible, typically almost colorless film was carefully peeled away and exhibited no oiliness or inhomogeneity unless otherwise noted. The film was cut into small pieces to be used for preparation of test bars by compression molding (size of pieces was similar to the hole dimensions of the mold plate). The film pieces were stacked into the holes of a multi-hole steel mold plate, pre-heated to 170° C., having hole dimensions 20 mm×12.8 mm×1.8 mm (ASTM D1693-95 dimensions). The mold plate was pressed in a PHI company QL-433-6-M2 model hydraulic press equipped with separate heating and cooling platforms. The upper and lower press plates were covered in Teflon™-coated aluminum foil and the following multistage press procedure was used at 170° C. with no release between stages: (1) 3 minutes with 1-2 ton overpressure; (2) 1 minute at 10 tons; (3) 1 minute at 15 tons; (4) 3 minutes at 30 tons; (5) release and 3 minutes in the cooling stage of the press (7° C.) at 30 tons. A knockout tool was then used to remove the sample bars with minimal flexion. Typically near-colorless, flexible bars were obtained which, when stored at room temperature, showed no oiliness or exudation several weeks after pressing unless otherwise noted.

### Example #19

### Initial and Room Temperature-Aged Clarity and Appearance of Plasticized PVC Bars

Two each of the sample bars prepared in Example 18 were visually evaluated for appearance and clarity and further compared to identically prepared bars plasticized with DINP by placing the bars over a standard printed text. The qualitative and relative flexibility of the bars was also crudely evaluated by hand. The various bars were evaluated in different test batches; thus, a new DINP control bar was included with each batch. The bars were placed in aluminum pans which were then placed inside a glass crystallization dish covered with a watch glass. The bars were allowed to sit under ambient conditions at room temperature for at least three weeks and re-evaluated during and/or at the end of this aging period. Table 3 below presents appearance rankings and notes for the ester-containing bars and the control DINP-containing bars.

TABLE 3

| | Initial | Final Clarity | |
|---|---|---|---|
| Ex. No. | Clarity | Value (day of | |
| (Plasticizer Used in Bar) | Value* | evaluation) | Notes on Bar** |
| 6 | 1^{a} | 1 (29) | Slightly stiff |
| 6 (repeat testing) | 1^{c} | 1 (26) | OK flex, slightly < DINP (day 26) |
| 10 | 1^{b} | 1.5 (25) | Good flex > DINP (day 24) |
| 11 | 1a | 1 (29) | Stiff |
| 13 | 1.5^{b} | 1.5 (25) | Brittle |
| 14 | 1^{c} | 1 (26) | Good/OK flex, ~DINP (day 26) |
| 15 | 1^{c} | 1 (26) | Good flex > DINP (day 26) |
| 16 | 1^{c} | 1 (26) | Ok flex, slightly < DINP (day 26) |
| DINP ctrl for 6, 11 | 1^{a} | 1 (29) | Not recorded |
| DINP ctrl for 10, 13 | 1^{b} | 1 (25) | Good flexibility |
| DINP ctrl for 6 (rpt), 14-16 | 1^{c} | 1 (26) | Good/Ok flex (day 26) |

*1-5 scale, 1 = no distortion, 5 = completely opaque.
**No bars exhibited oiliness, stickiness, or inhomogeneity unless otherwise noted.
^{a}Evaluated 3 days after pressing.
^{b}Evaluated 7 days after pressing.
^{c}Evaluated 2 days after pressing.

### Example #20

### 98° C. Weight Loss Properties of Plasticized PVC Bars

Two each of the PVC sample bars prepared in Example 18 were placed separately in aluminum weighing pans and placed inside a convection oven at 98° C. Initial weight measurements of the hot bars and measurements taken at specified time intervals were recorded and results were averaged between the bars. The averaged results are shown in Table 4. Notes on the appearance and flexibility of the bars at the end of the test are also given. The final color of the bars (even DINP control samples) varied between batches; gross comparisons only should be made between bars of different test batches.

TABLE 4

| Example No. | | | | | | | |
|---|---|---|---|---|---|---|---|
| (Plasticizer | Day | Day | Day | Day | Day | Day | |
| Used in Bar) | 1 | 2 | 3 | 7 | 14 | 21 | Notes on Bar* |
| 6 | 0.28 | 0.36 | 0.39 | 0.45 | 0.57 | 0.60 | Med brown, |
| | | | | | | | flex > DINP |
| 6 (repeat) | 0.12 | — | — | 0.31 | 0.43 | 0.58 | Med orange, still good flex |
| | | | | | | | (>DINP) |
| 10 | 0.28 | 0.41 | 0.45 | 0.66 | 1.04 | 1.53 | Light brown, |
| | | | | | | | flex > DINP |
| 11 | 0.27 | 0.31 | 0.32 | 0.37 | 0.51 | 0.52 | Med brown, oily, |
| | | | | | | | fairly brittle |
| 13 | 0.76 | 0.83 | 0.78 | 0.89 | 0.89 | 0.92 | Oily, med-dark brown, brittle |
| 14 | 0.11 | — | — | 0.29 | 0.41 | 0.50 | Clear, minor darkening, still |
| | | | | | | | good flex (>DINP) |
| 15 | 0.13 | — | — | 0.25 | 0.35 | 0.47 | Clear, burn spots, very good |
| | | | | | | | flex (>DINP) |
| 16 | 0.15 | — | — | 0.26 | 0.42 | 0.48 | Clear to light orange, very |
| | | | | | | | good flex (>DINP) |
| DINP ctrl for | 0.26 | 0.33 | 0.40 | 0.55 | 0.73 | 0.83 | Med brown, |
| 6, 11 | | | | | | | OK flex |
| DINP ctrl for | 0.21 | 0.22 | 0.24 | 0.37 | 0.56 | 0.60 | Light brown, |
| 10, 13 | | | | | | | flexible |
| DINP ctrl for 6 | 0.17 | — | — | 0.48 | 0.67 | 0.94 | Medium orange, still good |
| (rpt), 14-16 | | | | | | | flex |

*No bars exhibited oiliness, stickiness, or inhomogeneity unless otherwise noted.

### Example #21

### 70° C. Humid Aging Clarity Properties of Plasticized PVC Bars

Using a standard one-hole office paper hole punch, holes were punched in two each of the sample bars prepared in Example 18 about ⅛″ from one end of the bar. The bars were hung in a glass pint jar (2 bars per jar) fitted with a copper insert providing a stand and hook. The jar was filled with about ½″ of distilled water and the copper insert was adjusted so that the bottom of each bar was about 1″ above the water level. The jar was sealed, placed in a 70° C. convection oven, and further sealed by winding Teflon® tape around the edge of the lid. After 21 days the jars were removed from the oven, allowed to cool for 20 minutes, opened, and the removed bars were allowed to sit under ambient conditions in aluminum pans (with the bars propped at an angle to allow air flow on both faces) or hanging from the copper inserts for about 1 week (until reversible humidity-induced opacity had disappeared). The bars were evaluated visually for clarity. All bars exhibited complete opacity during the duration of the test and for several days after removal from the oven. Results are shown in Table 5. Notes on the appearance and flexibility of the bars at the end of the test are also given.

TABLE 5

| | Clarity Value | |
|---|---|---|
| Example No. | After Test (Days | |
| (Plasticizer Used in Bar) | aged at ambient) | Notes on Bar** |
| 6 | 1.5 (10) | Still relatively flexible |
| 6 (repeat) | 1 (12) | Ok flex ~DINP |
| 10 | 1 (8) | Good flex > DINP |
| 11 | 2 (10) | Oily, fairly brittle |
| 13 | 2 (8) | Oily, very brittle |
| 14 | 1 (12) | Good flex > DINP |
| 15 | 1 (12) | Good/OK flex, sl. > |
| | | DINP |
| 16 | 1 (12) | Ok flex ~DINP, |
| | | v. minor oiliness |
| DINP ctrl for 6, 11 | 1.5 (10) | Still very flexible |
| DINP ctrl for 10, 13 | 1 (8) | Somewhat flexible |
| DINP ctrl for 6 (rpt), 14-16 | 1-1.5 (12) | Ok flex, some residual |
| | | opacity |

*1-5 scale, 1 = no distortion, 5 = completely opaque.
**No bars exhibited oiliness, stickiness, or inhomogeneity unless otherwise noted; these qualities may represent incomplete reversal of humidity-induced opacity.

### Example #22

### Calorimetric Weight Loss Study of Plasticized PVC Bars

A small portion of selected plasticized sample bars prepared in Example 18 were subjected to Thermogravimetric Analysis as previously described to evaluate plasticizer volatility in the formulated test bars. Results are shown in Table 6.

TABLE 6

| Ex. No. of Material | TGA 1% | TGA 5% | TGA 10% | % Loss, |
|---|---|---|---|---|
| Used in Bar | Loss (° C.) | Loss (° C.) | Loss (° C.) | 220° C. |
| DINP | 204.6 | 247.4 | 257.6 | 1.8 |
| 6^{a} | 205.8 | 241.1 | 252.4 | 2.0 |
| | (214.5) | (246.9) | (2.0) | (1.3) |
| 11^{b} | 212.0 | 243.3 | 255.2 | 1.4 |
| | (209.3) | (247.4) | (257.9) | (1.6) |
| 14 | 215.7 | 247.2 | 257.9 | 1.2 |
| 15 | 222.4 | 251.0 | 263.2 | 0.9 |
| 16 | 217.8 | 250.9 | 262.1 | 1.1 |

^{a}First values are for a film aged 491 days, parenthetical values are for a bar aged 9 days.
^{b}First values are for a film aged 493 days, noted as oily at time of analysis; parenthetical values are for a bar aged 8 days.

### Example #23

### Demonstration of PVC Plasticization by Differential Scanning Calorimetry (DSC) and Dynamic Thermal Mechanical Analysis (DMTA)

Three-point bend Dynamic Mechanical Thermal Analysis (DMTA) with a TA Instruments DMA Q980 fitted with a liquid N₂ cooling accessory and a three-point bend clamp assembly was used to measure the thermo-mechanical performance of neat PVC and the PVC/plasticizer blend sample bars prepared in Example 18. Samples were loaded at room temperature and cooled to −60°-−90° C. at a cooling rate of 3° C./min. After equilibration, a dynamic experiment was performed at one frequency using the following conditions: 3° C./min heating rate, 1 Hz frequency, 20 micrometer amplitude, 0.01 pre-load force, force track 120%. Two or three bars of each sample were typically analyzed; numerical data was taken from the bar typically exhibiting the highest room temperature storage modulus (the bar assumed to have the fewest defects) unless another run was preferred for data quality. Glass transition onset values were obtained by extrapolation of the tan delta curve from the first deviation from linearity. The DMTA measurement gives storage modulus (elastic response modulus) and loss modulus (viscous response modulus); the ratio of loss to storage moduli at a given temperature is tan delta. The beginning (onset) of the T_{g} (temperature of brittle-ductile transition) was obtained for each sample by extrapolating a tangent from the steep inflection of the tan delta curve and the first deviation of linearity from the baseline prior to the beginning of the peak. Table 7 provides a number of DMTA parameters for the bars (the temperature at which the storage modulus equals 100 MPa was chosen to provide an arbitrary measure of the temperature at which the PVC loses a set amount of rigidity; too much loss of rigidity may lead to processing complications for the PVC material). The flexible use temperature range of the samples was evaluated as the range between the T_{g} onset and the temperature at which the storage modulus was 100 MPa. A lowering and broadening of the glass transition for neat PVC was observed upon addition of the ester plasticizers, indicating plasticization. Plasticization (enhanced flexibility) was also demonstrated by lowering of the PVC room temperature storage modulus. Differential Scanning calorimetry (DSC) was also performed on the compression-molded sample bars (−90° C. to 100-170° C. at 10° C./min). Small portions of the sample bars (˜5-7 mg) were cut for analysis, making only vertical cuts perpendicular to the largest surface of the bar to preserve the upper and lower compression molding “skins”; the pieces were then placed in the DSC pans so that the upper and lower “skin” surfaces contacted the bottom and top of the pan. Alternately, DSC was conducted on leftover pieces of thin film. Results are summarized in Table 7; lowering and broadening of the glass transition for neat PVC indicates plasticization by the esters (for aid in calculating the numerical values of these broad transitions, the DSC curve for each plasticized bar or film was overlaid with the analogous DMTA curve for guidance about the proper temperature regions for the onset, midpoint, and end of T_{g}).

TABLE 7

| | Tan Δ | | 25° C. | Temp. of | Flex. | DSC | DSC | DSC | |
|---|---|---|---|---|---|---|---|---|---|
| Ex. No. | T_{g} | Tan Δ | Storage | 100 MPa | Use | T_{g} | T_{g} | T_{g} | Tₘ Max |
| of Mat. | Onset | Peak | Mod. | Storage | Range | Onset | Midpt | End | (° C.), |
| in Bar | (° C.) | (° C.) | (MPa) | Mod. (° C.) | (° C.)^{a} | (° C.) | (° C.) | (° C.) | ΔH_{f} (J/g)^{b} |
| DINP | −37.6 | 17.1 | 48.6 | 16.9 | 54.5 | −37.8 | −24.8 | −12.2 | N/A^{d} |
| 6^{e} | −48.2 | 22.2 | 48.1 | 16.6 | 64.8 | −52.8 | −36.5 | −20.2 | 62.8, 1.6 |
| | | | | | | (−55.0) | (−31.8) | (−8.8) | (55.6, 0.72) |
| 11^{f} | −49.2 | 37.6 | 100.9 | 25.1 | 74.3 | −59.0 | −42.0 | −25.1 | 63.0, 1.9 |
| | | | | | | (−53.8) | (−38.9) | (−23.6) | (55.7, 1.0) |
| 14 | −48.2 | 18.5 | 74.1 | 20.9 | 69.1 | −58.9 | −43.3 | −27.7 | 54.1 (0.48) |
| 15 | −48.8 | 3.0 | 25.9 | 3.2 | 52.0 | −48.0 | −23.5 | 0.8 | 57.3 (0.93) |
| 16 | −47.0 | 14.0 | 33.3 | 10.3 | 57.3 | −49.7 | −26.8 | −4.0 | 57.6 (1.10) |
| None^{c} | 44.0 | 61.1 | 1433 | 57.1 | 13.1 | 44.5 | 46.4 | 48.9 | N/A |

N/A = Not analyzed.
^{a}Difference between DMTA temperature of 100 MPa storage modulus and onset of T_{g}.
^{b}Some sample bars showed a weak melting point (Tₘ) from the crystalline portion of PVC. Often this weak transition was not specifically analyzed, but data is given here in instances where it was recorded.
^{c}Neat PVC, no plasticizer used.
^{d}Very small.
^{e}DSC First values are for a bar aged 499 days; parenthetical values are for a film aged 9 days; DMTA values are for a bar aged 16/44 days.
^{f}DSC first values are for a film aged 498 days, noted as oily at time of analysis; parenthetical values are for a bar aged 9 days. Film showed a second T_{g} at onset 7.2° C., midpt 11.1° C., end 14.8 C.; DMTA values are for a bar aged 16/31 days.

### Example #24

### Further Demonstration of PVC Plasticization with Ester Plasticizers

A plasticized PVC sample was prepared by first adding to 200 grams of OXO 240 PVC polymer, 5 grams of Therm-Check SP 175 stabilizer, 4 grams of Drapex 6.8 epoxidized soybean oil, 0.4 grams of stearic acid and 100 grams of the plasticizing ester of Example #6. This mixture was milled on a Dr. Collins 2 roll mill at 335° F. for 6 minutes and then removed. After cooling the samples were compression molded at 345° F. into standard 6 inch by 6 inch coupons and evaluated. The plasticizing ester of Example #6 gave a Shore A (15 second) hardness of 82.1, a 100% modulus of 1690 psi, ultimate tensile strength of 3229 psi, and ultimate elongation of 346%. After aging die cut dumbell specimens for 7 days at 100° C., in an oven with 150 air changes/hour, the 100% modulus had increased to 1998 psi, the tensile strength remained unchanged at 3212 psi, and the elongation was 323%. The sample specimens lost 3.9% by weight. Carbon volatile losses in the carbon volatility test were 0.5%. Compatibility of the plasticizer with the PVC was estimated through ⅜ in loop test and through 100% relative humidity testing at 70° C. for up to 21 days. No evidence of hydrolysis nor plasticizer incompatibility was observed. Performance advantage of this inventive plasticizer over that of DINP included reduced weight loss, increased elongation, and increased elongation after aging. Plasticizing efficiency as determined by Shore A hardness was equal to DINP.

A second plasticized PVC sample was prepared by first adding to 200 grams of OXO 240 PVC polymer, 5 grams of Therm-Check SP175 stabilizer, 4 grams of Drapex 6.8 epoxidized soybean oil, 0.4 grams of stearic acid and 100 grams of the plasticizing ester of Example #14. This mixture was milled on a Dr. Collins 2 roll mill at 335° F. for 6 minutes and then removed. After cooling the samples were compression molded at 345 F into standard 6 inch by 6 inch coupons (see above) and evaluated. The plasticizing ester of Example #14 gave a Shore A (15 second) hardness of 81.6, a 100% modulus of 1621 psi, ultimate tensile strength of 2964 psi, and ultimate elongation of 315%. After aging die cut dumbell specimens for 7 days at 100° C. in an oven with 150 air changes/hour, the 100% modulus had increased to 1812 psi, the tensile strength at 3149 psi, and the elongation was 332%. The sample specimens lost 2.8% by weight. Carbon volatile losses in the carbon volatility test were 0.5%. Compatibility of the plasticizer with the PVC was estimated through ⅜ in loop test and through 100% relative humidity testing at 70 C for up to 21 days. No evidence of hydrolysis nor plasticizer incompatibility was observed. Performance advantages of this inventive plasticizer over that of DINP included reduced weight loss and increased elongation after aging. Plasticizing efficiency as determined by Shore A hardness was slightly better than DINP.

A plasticized PVC sample was prepared by first adding to 200 grams of OXO 240 PVC polymer, 5 grams of Therm-Check SP175 stabilizer, 4 grams of Drapex 6.8 epoxidized soybean oil, 0.4 grams of stearic acid and 100 grams of the plasticizing ester of example #12. This mixture was milled on a Dr. Collins 2 roll mill at 335° F. for 6 minutes and them removed. After cooling the samples were compression molded at 345° F. into standard 6 inch by 6 inch coupons and evaluated. The plasticizing ester of Example #15 gave a Shore A (15 second) hardness of 81.0, a 100% modulus of 1692 psi, ultimate tensile strength of 3111 psi, and ultimate elongation of 322%. After aging die cut dumbell specimens for 7 days at 100° C., in an oven with 150 air changes/hour, the 100% modulus had increased to 1763 psi, the tensile strength remained unchanged at 3136 psi, and the elongation was 342%. The sample specimens lost 1.8% by weight. Carbon volatile losses in the carbon volatility test were 0.5%. Compatibility of the plasticizer with the PVC was estimated through ⅜ in loop test and through 100% relative humidity testing at 70° C. for up to 21 days. No evidence of hydrolysis nor plasticizer incompatibility was observed. Performance advantage of this inventive plasticizer over that of DINP included reduced weight loss, increased elongation, and increased elongation after aging. Plasticizing efficiency as determined by Shore A hardness was better than DINP.

A plasticized PVC sample was prepared by first adding to 200 grams of OXO 240 PVC polymer, 4 grams of Nafsafe PKP314 stabilizer, 0.4 grams of stearic acid, 40 grams of calcium carbonate and 120 grams of the plasticizing ester of Example #12. This mixture was milled on a Dr. Collins 2 roll mill at 335 F for 6 minutes and them removed. After cooling the samples were compression molded at 345° F. into standard 6 inch by 6 inch coupons and evaluated. The plasticizing ester of Example #12 gave a Shore A (15 second) hardness of 80.5, a Shore D hardness of 23.4, and ultimate tensile strength of 3091 psi, 100% modulus of 157 s pis, and ultimate elongation of 367%. After aging die cut dumbell specimens for 7 days at 100° C., in an oven with 150 air changes/hour, the 100% modulus had increased to 1893 psi, the tensile strength decreased slightly to 2925 psi, and the elongation was 336%. The sample specimens lost 6.7% by weight. Carbon volatile losses in the carbon volatility test were 0.2%. Low temperature flexibility of this PVC formulation as determined by the Clash-Berg method gave a Tf value of −24.3° C. Compatibility of the plasticizer with the PVC was estimated through ⅜ in loop test and through 100% relative humidity testing at 70° C. for up to 21 days. No evidence of hydrolysis nor plasticizer incompatibility was observed. Performance advantage of this inventive plasticizer over that of DINP included reduced weight loss, increased elongation, increased elongation after aging, Plasticizing efficiency and low temperature flexibility as determined by Shore A hardness and Clash-Berg Tf were essentially equivalent to that recorded for DINP in the same formulation. UV exposure as determined by QUV exposure, type B bulbs, for 28 days found the inventive plasticizer of Example #12 has better color retention than DINP.

Plasticized PVC samples containing either the ester plasticizers of Example 6 or DINP (as a comparative) were mixed at room temperature with moderate stirring, then placed on a roll mill at 340° F. and milled for 6 minutes. The flexible vinyl sheet was removed and compression molded at 340° F. The samples had the following formulation: 100 phr Oxy Vinyls® 240 PVC resin; 50 phr oxo-ester or DINP; 2.2-2.5 phr epoxidized soybean oil; 2.5-3.3 phr Mark® 1221 Ca/Zn stabilizer; 0.3 phr stearic acid. Comparison of the data for the formulations is given in Table 8.

TABLE 8

| | Ex. 6 | Ex. 6 | |
|---|---|---|---|
| | C₉ 1,2-Ph Diester | C₉ 1,2-Ph Diester | |
| Plasticizer Used in Formulation | (aged 70° C.) | (aged 100° C.) | DINP |
| Original Mechanical Properties | | | |
| Shore A Hardness (15 sec.) | 78.9 | 80.8 | 82.7 |
| 95% Confidence Interval | 0.5 | 0.8 | 0.1 |
| Shore D Hardness (15 sec.) | 25.6 | — | — |
| 95% Confidence Interval | 0.3 | — | — |
| 100% Modulus Strength, psi | 1624 | 1569 | 1687 |
| 95% Confidence Interval | 20 | 31 | 14 |
| Ultimate Tensile Strength, psi | 3107 | 3072 | 3095 |
| 95% Confidence Interval | 105 | 69 | 97 |
| Ultimate Elongation, % | 364 | 357 | 356 |
| 95% Confidence Interval | 15 | 17 | 18 |
| | 70° C. | 100° C. | 100° C. |
| Aged Mechanical Properties | | | |
| (7 days at given temp., AC/hour) | | | |
| Aged 100% Modulus Strength, psi | 1662 | 1965 | 2568 |
| 95% Confidence Interval | 21 | 18 | 15 |
| Ultimate Tensile Strength, psi | 3073 | 2857 | 2983 |
| 95% Confidence Interval | 114 | 156 | 75 |
| Ultimate Elongation, % | 356 | 297 | 259 |
| 95% Confidence Interval | 23 | 35 | 9 |
| Weight Loss, Wt % | 0.3 | 5.0 | 10.1 |
| 95% Confidence Interval | 0.03 | 0.22 | 0.3 |
| Retained Properties | | | |
| (7 days at given temp., AC/hour) | | | |
| Retained 100% Modulus Strength, % | 102 | 125 | 152 |
| 95% Confidence Interval | 0.3 | 0.4 | 0.3 |
| Retained Tensile Strength, % | 99 | 93 | 96 |
| 95% Confidence Interval | 0.4 | 0.4 | 0.3 |
| Retained Elongation, % | 98 | 83 | 73 |
| 95% Confidence Interval | 1.4 | 1.6 | 1.0 |
| Low Temperature | | | |
| Clash Berg (T_{f}), ° C. | −23.7 | −27.7 | −18.0 |
| 95% Confidence Interval | 4.2 | 1.6 | 1.0 |

— = Data unavailable.

1. One or more phenylene oxo-diesters chosen from the following formulae: 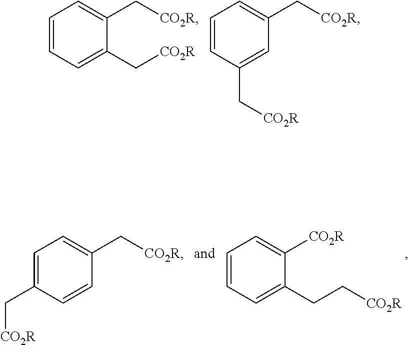
wherein each R is the alkyl residue of one or more C₄ to C₁₄ oxo-alcohols.

2. The one or more phenylene oxo-diesters of claim 1, wherein R has an average branching of from 0.2 to 4.0 branches per group.

3. A process for making 1,2-phenylene oxo-diesters, comprising:
selectively hydrogenating naphthalene to form a partially hydrogenated naphthalene; oxygenating said partially hydrogenated naphthalene to form phenylene diacids; and esterifying said phenylene diacids with oxo-alcohols to form 1,2-phenylene oxo-diesters.

4. The process of claim 3, wherein said 1,2-phenylene oxo-diesters are selected from the following formulae and mixtures thereof: 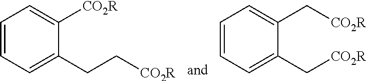
wherein each R is the alkyl residue of one or more C₄ to C₁₄ oxo-alcohols.

5. The process of claim 3, wherein said selective hydrogenation is conducted by reacting naphthalene with hydrogen at temperatures between 30° C. and 300° C., and a pressure of between 100 kPa to 2000 kPa to form tetralin.

6. The process of claim 3, wherein said selective hydrogenation is conducted by reacting naphthalene with hydrogen at temperatures between 30° C. and 300° C., and a pressure of between 100 kPa to 2000 kPa to form dihydronaphthalene.

7. The process of claim 5, wherein said oxidation of tetralin is conducted by reacting tetralin with an oxidant at temperatures between 30° C. and 300° C., to form 1,2-phenylene diacids.

8. The process of claim 6, wherein said oxidation of dihydronaphthalene is conducted by reacting dihydronaphthalene with an oxidant at temperatures between 30° C. and 300° C., to form 1,2-phenylene diacids.

9. The process of claim 3, wherein said esterification of 1,2-phenylene diacids is conducted by reacting said 1,2-phenylene diacids with C₄ to C₁₄ oxo-alcohols at temperatures between 100° C. and 250° C., to form 1,2-phenylene oxo-diesters.

10. The process of claim 4, wherein each R is C₄ to C₁₄ branched alkyl, or mixtures of linear and branched alkyl.

11. A process for making phenylene oxo-diesters, comprising:
selectively brominating xylene to form bisbromomethylbenzene; carboalkoxlating said bisbromomethylbenzene with a palladium catalyst to form dimethylphenylene diacetate; and transesterifying of said diphenylene diacetate with oxo-alcohols to form phenylene oxo-diesters.

12. The process of claim 11, wherein the xylene is o-xylene, the bisbromomethylbenzene is 1,2-bisbromomethylbenzene, and the phenylene oxo-diesters are 1,2-phenylene oxo-diesters.

13. The process of claim 11, wherein the xylene is m-xylene, the bisbromomethylbenzene is 1,3-bisbromomethylbenzene, and the phenylene oxo-diesters are 1,3-phenylene oxo-diesters.

14. The process of claim 11, wherein the xylene is p-xylene, the bisbromomethylbenzene is 1,4-bisbromomethylbenzene, and the phenylene oxo-diesters are 1,4-phenylene oxo-diesters.

15. The process of claim 11, wherein the xylene is a mixture of two or more of o-xylene, m-xylene, or p-xylene.

16. A polymer composition comprising a polymer and at least one phenylene oxo-diester selected from the following formulae and mixtures thereof: 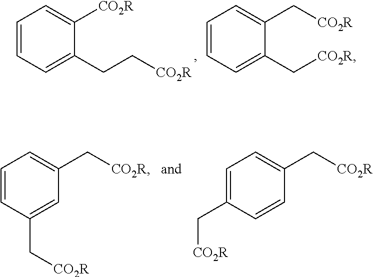
wherein each R is the alkyl residue of one or more C₄ to C₁₄ oxo-alcohols.

17. The polymer composition of claim 16, wherein the polymer is selected from the group consisting of vinyl chloride resins, polyesters, polyurethanes, ethylene-vinyl acetate copolymer, rubbers, poly(meth)acrylics and combinations thereof.

18. The polymer composition of claim 16, wherein R has an average branching of from 0.2 to 4.0 branches per group.
All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted.
When numerical lower limits and numerical upper limits are listed herein, ranges from any lower limit to any upper limit are contemplated. While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains.
The present invention has been described above with reference to numerous embodiments and specific examples. Many variations will suggest themselves to those skilled in this art in light of the above detailed description. All such obvious variations are within the full intended scope of the appended claims.

1. Oxo-diesters of the formula: 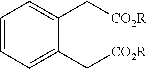
wherein each R is the alkyl residue of one or more C₄ to C₁₄ oxo-alcohols.

2. Oxo-diesters of the formula 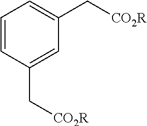
wherein each R is the alkyl residue of one or more C₄ to C₁₄ oxo-alcohols.

3. Oxo-diesters of the formula 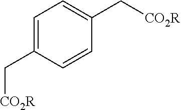
wherein each R is the alkyl residue of one or more C₄ to C₁₄ oxo-alcohols.

4. Oxo-diesters of the formula: 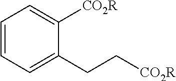
wherein each R is the alkyl residue of one or more C₄ to C₁₄ oxo-alcohols.

5. A mixture of two or more phenylene oxo-diesters chosen from the following formulae: 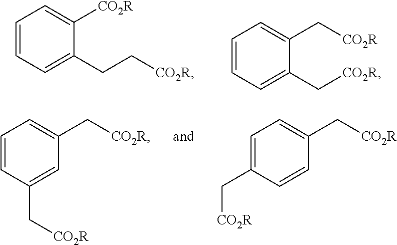
wherein each R is the alkyl residue of one or more C₄ to C₁₄ oxo-alcohols.

6. The oxo-diesters of any one of the preceding claims, wherein R has an average branching of from 0.2 to 4.0 branches per group.

7. A process for making 1,2-phenylene oxo-diesters, comprising:
selectively hydrogenating naphthalene to form a partially hydrogenated naphthalene;
oxygenating said partially hydrogenated naphthalene to form phenylene diacids; and
esterifying said phenylene diacids with oxo-alcohols to form 1,2-phenylene oxo-diesters.

8. The process of claim 7, wherein said 1,2-phenylene oxo-diesters are selected from the following formulae and mixtures thereof: 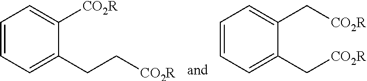
wherein each R is the alkyl residue of one or more C₄ to C₁₄ oxo-alcohols.

9. The process of claim 7, wherein said selective hydrogenation is conducted by reacting naphthalene with hydrogen at temperatures between 30° C. and 300° C., and a pressure of between 100 kPa to 2000 kPa to form tetralin.

10. The process of claim 7, wherein said selective hydrogenation is conducted by reacting naphthalene with hydrogen at temperatures between 30° C. and 300° C., and a pressure of between 100 kPa to 2000 kPa to form dihydronaphthalene.

11. The process of claim 9, wherein said oxidation of tetralin is conducted by reacting tetralin with an oxidant at temperatures between 30° C. and 300° C., to form 1,2-phenylene diacids.

12. The process of claim 10, wherein said oxidation of dihydronaphthalene is conducted by reacting dihydronaphthalene with an oxidant at temperatures between 30° C. and 300° C., to form 1,2-phenylene diacids.

13. The process of claim 7, wherein said esterification of 1,2-phenylene diacids is conducted by reacting said 1,2-phenylene diacids with C₄ to C₁₄ oxo-alcohols at temperatures between 100° C. and 250° C., to form 1,2-phenylene oxo-diesters.

14. The process of claim 8, wherein each R is C₄ to C₁₄ branched alkyl, or mixtures of linear and branched alkyl.

15. A process for making phenylene oxo-diesters, comprising:
selectively brominating xylene to form bisbromomethylbenzene;
carboalkoxlating said bisbromomethylbenzene with a palladium catalyst to form dimethylphenylene diacetate; and
transesterifying said diphenylene diacetate with oxo-alcohols to form phenylene oxo-diesters.

16. The process of claim 15, wherein the xylene is o-xylene, the bisbromomethylbenzene is 1,2-bisbromomethylbenzene, and the phenylene oxo-diesters are 1,2-phenylene oxo-diesters.

17. The process of claim 15, wherein the xylene is m-xylene, the bisbromomethylbenzene is 1,3-bisbromomethylbenzene, and the phenylene oxo-diesters are 1,3-phenylene oxo-diesters.

18. The process of claim 15, wherein the xylene is p-xylene, the bisbromomethylbenzene is 1,4-bisbromomethylbenzene, and the phenylene oxo-diesters are 1,4-phenylene oxo-diesters.

19. The process of claim 15, wherein the xylene is a mixture of two or more of o-xylene, m-xylene, or p-xylene.

20. A polymer composition comprising a polymer and at least one phenylene oxo-diester selected from the following formulae and mixtures thereof: 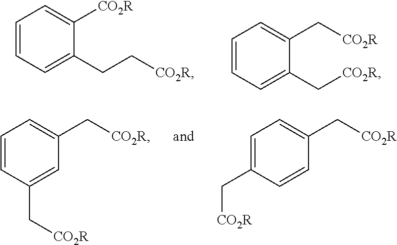
wherein each R is the alkyl residue of one or more C₄ to C₁₄ oxo-alcohols.

21. The polymer composition of claim 20, wherein the polymer is selected from the group consisting of vinyl chloride resins, polyesters, polyurethanes, ethylene-vinyl acetate copolymer, rubbers, poly(meth)acrylics and combinations thereof.

22. The polymer composition of claim 21, wherein the polymer is a polymer blend of polyvinyl chloride with an ethylene-vinyl acetate copolymer.

23. The polymer composition of claim 21, wherein the polymer is a polymer blend of polyvinyl chloride with a polyurethane.

24. The polymer composition of claim 21, wherein the polymer is a polymer blend of polyvinyl chloride with an ethylene-based polymer.

25. The polymer composition of claim 21, wherein said polymer is polyvinyl chloride.

26. The polymer composition of claim 20, wherein R has an average branching of from 0.2 to 4.0 branches per group.
